(19) United States
(12) Patent Application Publication
(10) Pub. No.: US 2017/0035825 A1
(43) Pub. Date: Feb. 9, 2017

(54) FOOD SUPPLEMENT COMPOSITION COMPRISING A CUCUMBER (CUCUMIS SATIVUS) EXTRACT

(71) Applicant: NATURALEA SA, Vezia (CH)

(72) Inventor: Robert Nash, Vezia (CH)

(21) Appl. No.: 15/301,680

(22) PCT Filed: Mar. 23, 2015
(86) PCT No.: PCT/IB2015/052115

§ 371 Date: Oct. 3, 2016

(30) Foreign Application Priority Data
Apr. 3, 2014 (GB) 1406026.3

(51) Int. Cl. A61K 36/42; A23L 33/105; A61K 9/00

(52) U.S. Cl. CPC A61K 36/42; A61K 9/0056; A23L 33/105; A23V 2002/00

(57) ABSTRACT

There is described a food supplement composition comprising a cucumber (Cucumis sativus) extract which in particular reduces the TNF-α level. Raw cucumber extract can inhibit the production of TNF-α in human blood without causing adverse immune reactions. It has therefore been shown that raw cucumber extract can be effectively used as food supplement product or as dietary supplement for a prolonged time for the prevention and treatment of TNF-α level dependant inflammation states.

### CROSS-REFERENCE TO RELATED APPLICATIONS

This application claims priority of International Application No. PCT/IB2015/052115, entitled FOOD SUPPLEMENT COMPOSITION COMPRISING A CUCUMBER (CUCUMIS SATIVUS) EXTRACT, filed Mar. 23, 2015, the entire contents of which is being incorporated by reference herein, which claims priority to British Patent Application No. 1406026.3, filed May 22, 2015.

### TECHNICAL FIELD

This invention relates to a food supplement composition comprising a raw extract from cucumber (Cucumis sativus) fruits, in particular to be used to reduce TNF-alpha and for the treatment of TNF-alpha mediated diseases.

### BACKGROUND

The cucumber (Cucumis sativus) is a widely cultivated plant in the gourd family.

Cucumbers and cucumber extracts have long been recognized as having anti-inflammatory properties, and have been used topically for various types of skin problems, including swelling under the eyes and sunburn. Cucumber was very popular in the ancient civilizations of Egypt, Greece and Rome, where it was used not only as a food, but also for its skin healing properties.

It has long been recognized that the anti-inflammatory properties may be related with the presence of iminosugar acids derived from many different plant sources.

Iminosugar acids (ISAs) constitute a subclass of the more widely distributed class of phytochemicals known as iminosugars. Many known ISAs are phytochemicals, present as secondary metabolites in plant tissues (where they may play a role in defense). While iminosugars are widely distributed in plants (Watson ef a/. (2001) Phytochemistry 56: 265-2951), the iminosugar acids are much less widely distributed.

WO2009103953 describes compositions comprising imino sugar acids for the treatment of energy utilization disease (e.g. metabolic syndrome, including any disease or disorder associated therewith, for example central obesity, elevated levels of triglycerides and diabetes, including type 1 diabetes, type 2 diabetes and insulin resistance), processes for producing said compositions from various plant sources including Stevia spp., Gymnema spp. (for example Gymnema sylvestre), Citrus spp., Aspalanthus linearis (Rooibos), Glycine max (Soya), Pumpkin {Cucurbita ficifolia and other Cucubitaceae species, e.g. Momordica charantia), Lycium barbarum (Goji) or mixtures thereof, together with various products, compounds, compositions, medical uses and methods based thereon.

WO2013054070 describes compositions comprising ido-BR1 isolated from Cucumis sativus and used in therapy or prophylaxis, including the treatment of inflammatory diseases and to reduce inflammation.

In particular according to this patent application the isolated iminosugar acid ido-BR1 named (2R,3R,4R,5S)-3,4,5-trihydroxypiperidine-2-carboxylic acid (ido-BR1) of formula:

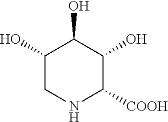 [SMILES: O=C(O)[C@@H]1NC[C@H](O)[C@@H](O)[C@@H]1O]

is an effective anti-inflammatory agent.

The efficacy of isolated ido-BR1 as an anti-inflammatory agent has been tested in a model of mucosal inflammation.

According to the experiments shown in the above patent application, the isolated ido-BR1 abrogated inflammation in the uterus explants. The abrogation of inflammation by isolated ido-BR1 occurred in a dose-dependent manner. However, the effect, although showing a similar pattern, was not significant at 72 h.

The patent application shows that the ido-BR1 has to be isolated with a very complex method starting with an extraction of cucumber fruits in water or aqueous solvents such as 70% aq. ethanol. Subsequently Cation exchange resins such as IR120 or Dowex 50 resin in the H<+> form (or similar form selected by those practiced in the art) will provide a concentrated fraction containing ido-BR1 along with amino acids. Use of an anion exchange resin such as CG400 or Dowex 1 in the OH form (or similar form selected by those practiced in the art) will also provide an ido-BR1-enriched fraction. All samples from ion exchange columns were freeze dried before derivatization.

Several approaches are nowadays known to suppress inflammation, the two most routinely used therapeutically at present are a) steroids (glucocorticoid analogues) and b) agents that neutralize the actions of TNF-α such as Etanercept (soluble TNF-α receptor fusion protein) and Infliximab (monoclonal antibody to TNF-α). Both these approaches target pro-inflammatory cytokines, suppression of production by steroids and preventing the actions of TNF-α by Etanercept and Infliximab. This also demonstrates the importance of targeting TNF-α to suppress inflammatory responses as shown in Feldmann M, Maini R N (2001). Anti-TNF alpha therapy of rheumatoid arthritis: what have we learned? Annual review of immunology 19: 163-196. Similarly, glucocorticoid analogues also suppress the production of cytokines and Dexamethasone, specifically, can reduce the LPS-stimulated production from human monocytes as shown in Waage A, Bakke O (1988). Glucocorticoids suppress the production of tumour necrosis factor by lipopolysaccharide-stimulated human monocytes. Immunology 63: 299-302.

It would be advantageous to have an alternative and more effective and efficient active principle able to suppress proinflammatory cytokine production.

Namely to date there is no reliable composition which could be used to suppress pro-inflammatory cytokine production for a prolonged time.

The use of anti-TNF therapies such as the soluble TNF-α receptor Etanercept and the anti-TNF-α antibody Infliximab, has demonstrated that targeting TNF-α in particular is a very effective treatment even in intractable diseases such as Crohn's disease.

These treatments titrate down the actions of TNF-α which is already circulating.

However, immune reactions to these anti-TNF-α proteins severely limits their prolonged use as shown in several studies and in particular:

Anti-TNF-alpha biotherapies: perspectives for evidence-based personalized medicine. Immunotherapy 4: 1167-1179; Steenholdt C, Svenson M, Bendtzen K, Thomsen O O, Brynskov J, Ainsworth M A (2012). Acute and delayed hypersensitivity reactions to infliximab and adalimumab in a patient with Crohn's disease. Journal of Crohn's & colitis 6: 108-111; van Schouwenburg P A, Rispens T, Wolbink G J (2013). Immunogenicity of anti-TNF biologic therapies for rheumatoid arthritis. Nature reviews. Rheumatology 9: 164-172.

It would be particularly advantageous to have an active principle able to reduce TNF-α levels for a prolonged time.

In particular, it would be advantageous to have a composition able to treat a subject for a prolonged time to titrate down the actions of TNF-α which is already circulating in the blood.

It is essential that the prolonged use does not cause immune reactions, for instance in cases of long-lasting inflammation states as can occur in sportsmen or chronic patients.

From Latin American Journal of Pharmacy, vol 29, No 6, 2010, pages 927-932, SINGH et al. “Antioxidant, Anti-inflammatory and analgesic potential of Cucumis sativus extract” is known the use of a methanolic seed extract (MECS). The Article specifies that for a potential use as anti-inflammatory or antioxidants the seeds should be considered and not the fruits. The only specific use shown is in the treatment of paw edema. The use is as single dose.

CN1742777 discloses a pharmaceutical composition comprising cucumber plant extract useful in the treatment of hepatitis, protecting the liver and reducing transaminase. This document does not disclose the use of cucumber fruits. Moreover this document does not disclose the use for the treatment of TNF mediated diseases and does not disclose any specific use as anti-inflammatory.

CN1184153 discloses an health drink comprising a juice of cucumber. No specific therapeutic use is indicated, no prolonged use is reported.

CN1058831 discloses a cucumber facial cleanser prepared by griding Frucuts Cucumidis Sativi extract of water and oil phases. Only a cosmetic use is indicated and no oral assumption is also indicated.

### SUMMARY

A first scope of the present invention is to provide for a composition able to treat a subject for a prolonged time to titrate down the actions of TNF-α which is already circulating in the blood without causing immune reactions.

According to the present invention said scope is reached by a food supplement composition according to this disclosure and its appended claims.

### BRIEF DESCRIPTION OF THE DRAWINGS

FIG. 1 shows a concentration-dependent action of LPS on TNF-α production in whole human blood.

FIG. 2 shows raw cucumber extract added at the same time as LPS (10 μg/ml) and incubated for 20 hr with reduced TNF-α production.

FIG. 3 shows a LPS induced spindle-shaped morphological change (stress fiber induction) and cell detachment Gherkin extract inhibited LPS-induced morphological change and cell detachment in a dose dependent manner.

FIG. 4 shows gherkin extract induced HO-1 expression in Gherkin treated cells.

FIG. 5 shows gherkin extract inhibited LPS-induced TNF-alpha expression.

FIG. 6 shows a graph reporting a score given by 20 athletes after a 4 week treatment.

FIG. 7 shows a graph reporting gherkin effects for 50 subjects.

FIG. 8 shows graphs representing inhibition of LPS-induced cytokines released by gherkin treated mouse dendritic cells.

FIG. 9 shows graphs of inhibition of LPS-induced cytokines released by gherkin treated human dendritic cells.

### DETAILED DESCRIPTION

It has now been surprisingly found that raw cucumber extract can inhibit the production of TNF-α in human blood without causing immune reactions.

In particular raw cucumber extract can be effectively used as a food supplement product or dietary supplement for a prolonged time without causing immune reactions.

The term “prolonged use” or for a “prolonged time” as applied to the composition of the invention is used herein to indicate that the composition of the invention may be administered for more than a week without causing an immune reaction contrarily to the aforementioned known products.

For more than one week is used herein to indicate that the composition is administered at list once a day, each day and for more than one week.

The administration is preferably an oral administration.

The term “isolated” as applied to the ido-BR1 is used herein to indicate that the ido-BR1 exists in a physical milieu distinct from that in which it occurs in nature (or in the case of synthetic ido-BR1 is purified to some degree) and is isolated with respect to the complex cellular milieu in which it naturally occurs (or with respect to the some or all of the starting products, intermediates, buffers, solvents, reactants and/or co-products from which it is synthesised).

The term “raw extract” is used to describe a water-extract, an aqueous ethanol extract (for example 50% water/ethanol) or a pressed and filtered juice (usually subsequently dried) without isolating any single active principle.

In particular, in contrast to the above definition of “isolated” compound, the “raw extract” refers to the complex cellular physical milieu as present in the original botanical source.

The term herbal medicine is used herein to define a pharmaceutical composition in which at least one active principle is not chemically synthesized and is a phytochemical constituent of a plant. In the present case, this non-synthetic active principle is not isolated (as defined above), but present together with other phytochemicals with which it is associated in the source plant. In the present case the herbal medicine comprises a more or less raw extract, infusion or fraction of a plant.

The term food supplement composition is used herein to define a food product which provides physiological benefits or protects or prevents against disease.

The term food supplement refers to concentrated sources of nutrients or other substances with a nutritional or physiological effect whose purpose is to supplement the normal diet. In other terms food supplement means any food the purpose of which is to supplement the normal diet and which is a concentrated source of a vitamin or mineral or other substance with a nutritional or physiological effect, alone or in combination. A food supplement is preferably sold in dose form. The definition of ‘to supplement’ can be interpreted as taken in addition to’ the diet.

The term dietary supplement is used as a synonym of food supplement.

The food supplement may be also administered to non human as pet food.

The food supplement of the present invention may comprise a food supplement composition as above defined.

The term “dose form” means a form such as capsules, pastilles, tablets, pills and other similar forms, sachets of powder, ampoules of liquids, drop dispensing bottles, and other similar forms of liquids or powders designed to be taken in measured small unit quantities.

The term “for the use in the reduction of the TNF-alpha level in a subject” means that the composition is administered to a subject to reduce the TNF-alpha level. The reduction of TNF-alpha level is beneficial in many cases.

The reduction of TNF-alpha level is beneficial not only in many TNF-alpha mediated inflammatory diseases, but also as a prevention in subjects that could develop such diseases as sportsmen with an high TNF alpha level after a training. In particular, the TNF-alpha mediated inflammatory disease may be one of adult respiratory distress syndrome, asthma, autoimmune disease, cachexia, a chronic pulmonary inflammatory disease, congestive heart failure, Crohn's disease, endotoxic shock, ENL in leprosy, fibrotic disease, graft rejection, hemodynamic shock, hyperoxic alveolar injury, inflammation, muscular inflammation, tendon inflammation, articular inflammation, malaria, meningitis, multiple sclerosis, mycobacterial infection, an oncogenic or cancerous condition, opportunistic infection in AIDS, osteoarthritis, post ischemic reperfusion injury, psoriasis, radiation damage, rheumatoid arthritis, rheumatoid spondylitis, septic shock, sepsis, sepsis syndrome, systemic lupus erythrematosis or ulcerative colitis.

The compound of the present invention is preferably in the form of unit.

The amount administered can vary widely according to the particular dosage unit employed, the period of treatment, the age and sex of the user and the particular compound selected.

Moreover, the compounds of the invention can be used in conjunction with other.

The compound can be administered with a pharmaceutical carrier using conventional dosage unit.

The preferred route of administration is oral administration.

In general a suitable dose will be in the range of 5 to 500, preferably 5 to 200 mg per kilogram body weight of the raw extract per day, preferably in the range of 10 to 150 mg per kilogram body weight per day and most preferably in the range 50 to 100 mg per kilogram body weight per day.

The desired dose is preferably presented as a single dose for daily administration for more than one week.

However, two or more sub-doses administered at appropriate intervals throughout the day may also be employed.

### Formulation

The Cucumis sativus raw extract may be formulated together with acceptable excipients.

The Cucumis sativus raw extract is preferably a fruit extract.

Any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc.

The food supplement compositions may take any suitable form, and include for example tablets, elixirs, capsules, solutions, suspensions, powders, granules and aerosols.

For oral administration the compound can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

Tablets for oral use may include the compound for use according to the invention, mixed with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound for use according to the invention is mixed with a solid diluent, and soft gelatin capsules wherein the active ingredient is mixed with water or an oil such as peanut oil, liquid paraffin or olive oil.

The compounds of the invention are preferably tableted with conventional tablet bases such as lactose, sucrose, and cornstarch in combination with binders such as acacia, cornstarch, or gelatin, lubricants intended to improve the flow of tablet granulations and to prevent the adhesion of tablet material to the surfaces of the tablet dies and punches, for example, talc, stearic acid, or magnesium, calcium, or zinc stearate, dyes, colouring agents, and flavouring agents intended to enhance the aesthetic qualities of the tablets and make them more acceptable to the patient.

More preferably the tablets comprise between 10 to 500 mg raw extract of Cucumis sativus, more preferably between 50 to 200 mg.

Preferably, the tablets further comprise at least a substance chosen from the group consisting of silicon dioxide, cellulose, calcium phosphate, magnesium stearate.

More preferably the tablet comprise a nucleus comprising the raw extract and an external layer.

More preferably the external layer is a gastro-resistant layer.

Even more preferably the gastro-resistant layer comprise diluents, preferably comprising at least a substance chosen from the group consisting of microcrystalline cellulose, saccharose, corn starch, lactose and/or a mixture thereof.

Preferably the nucleus and/or the external layer comprises at least a physiologically acceptable excipient selected from suspending agents and/or glidants, filming agents, plasticizing agents and/or a mixture thereof.

Preferably the suspending agents and/or glidants are selected from colloidal anhydrous silica, talc and/or a mixture thereof.

Preferably the filming agents are selected from alkyl cellulose hydroxyalkyl cellulose, polyvinylpyrrolidone (PVP), polyvinyl alcohol (PVA) and/or a mixture thereof.

More preferably the filming agents is polyvinylpyrrolidone.

Preferably the plasticizing agents are selected from polyalkylene glycols, glycols and/or a mixture thereof.

More preferably the plasticizing agents are selected from polyethylene glycol (PEG) 400, polyethylene glycol (PEG) 6000, propylene glycol, triethyl citrate, triacetin and/or a mixture thereof.

Preferably, the external gastro-resistant layer comprises polymers and/or copolymers of acrylic acid and/or of methacrylic acid or salts.

More preferably the polymers and/or copolymers of acrylic acid and/or of methacrylic acid or salts gastro are selected from the copolymer of methacrylic acid type B, the copolymer of methacrylic acid type C and/or a mixture thereof.

Even more preferably said polymers and/or copolymers of acrylic acid and/or of methacrylic acid comprise sodium carboxymethylcellulose.

Preferably the suspending agents and/or glidants are selected from colloidal anhydrous silica, talc and/or a mixture thereof.

More preferably the tablet comprise also a whitening agent as titanium bioxide.

A suitable example of composition of a tablet comprises:
a nucleus of 500 mg weight comprising 150 mg of dried raw extract of Cucumis sativus comprising silicon dioxide, microcrystalline cellulose, calcium phosphate, magnesium stearate
an external gastroresistant layer comprising Shellac (E904), polyvinylpirrolidone, ammonium bicarbonate, Carboxymethyl cellulose sodium, medium-chain triglyceride, silicon bioxide, titanium bioxide (E171).

### EXEMPLIFICATION

The invention will now be described with reference to specific Examples. These are merely exemplary and for illustrative purposes only: they are not intended to be limiting in any way to the scope of the invention. These examples constitute the best mode currently contemplated for practicing the invention.

### Example 1

Blood and buffy coat fractions were supplied by the Scottish National Blood Transfusion Service (SNBTS), Glasgow, UK. Lipopolysaccharide (from Salmonella abortus equi) was purchased from Sigma-Aldrich Co. Ltd. (UK). PGE2 was from Cayman Chemical Co. (Ann Arbor, Mich.). Human TNF-α antibody pairs for TNF-α ELISA assays was from Invitrogen/Life Sciences Europe. All drugs were dissolved in RPMI 1640 medium from Gibco BRL, UK.

The cucumber (Cucumis sativus) extract was a 50 kg (batch 1) vacuum dried 50% aq. ethanol extract. The extract is an extract of the fruit of cucumber.

In experiments where whole blood incubations were carried out, blood was used without any further treatment following donation. Blood was kindly supplied by the Scottish National Blood Transfusion Service from normal healthy donors as defined by ensuring they all tested negative for HIV, hepatitis B & C, CMV and parasitic diseases such as malaria (as tested by the National Blood Transfusion service). They were also confirmed by our laboratory to be free of acute inflammatory disease at the time the blood was taken by measuring the “basal” level of TNF-α which was always <50 pg/ml.

Aliquots (800 μl) of whole blood were incubated with various concentrations of cucumber extract in RPMI 1640, as indicated in the results, LPS was added and incubations carried out for 20 hr at 37° C. in a humidified (100%) atmosphere of 5% CO2 in air. At the end of the incubation period, supernatants consisting of plasma were collected by centrifugation, 10,000 g for 30 seconds at room temperature and TNF-α levels measured using a human TNF-α ELISA system (BioSource Europe S.A., Belgium, supplied by Invitrogen), according to the manufacturer's instructions as described previously (Brown et al., 2013).

The data are expressed as mean values ±standard deviation. Results for TNF-α production are expressed as pg of TNF-α/ml blood.

Differences between control and treated groups were compared using multiple comparisons by Analysis of Variance (Fishers PLSD). P-values less than 0.05 were considered significant. All statistical analyses were carried out using Statview Software (Abacus Concepts, Inc. USA).

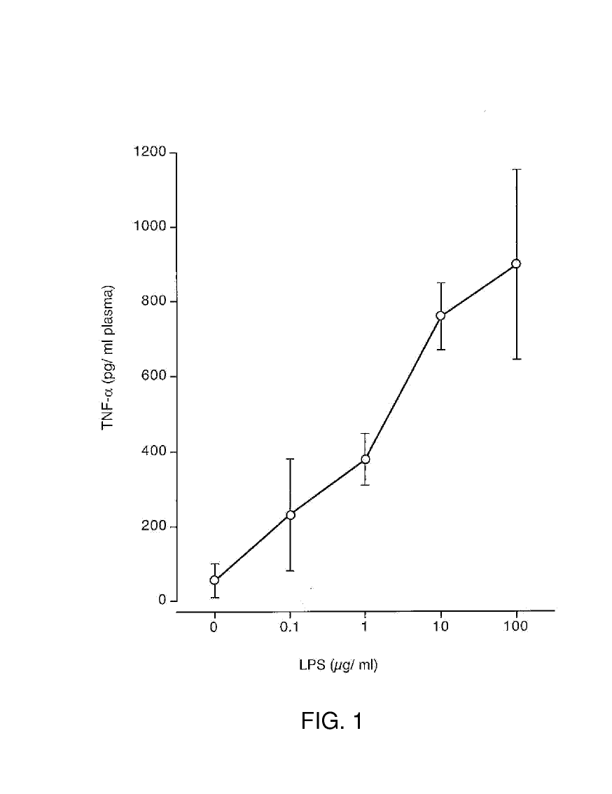

FIG. 1 shows the typical concentration-dependent action of LPS on TNF-α production in whole human blood and is typical of >100 separate experiments from our laboratory. In experiments where only a single concentration of LPS is required, 10 μg/ml is normally used as it stimulates a circa 80% stimulation providing opportunity for up- or down regulation in response to modulators.

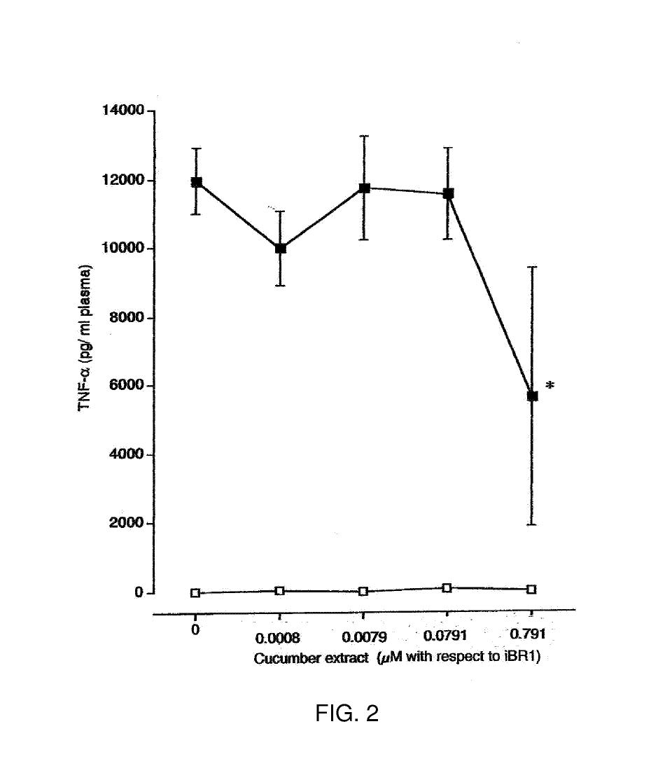

In FIG. 2 is shown that raw cucumber extract (which had been filtered using 0.22 μM filter units) added at the same time as LPS (10 μg/ml) and incubated for 20 hr reduced TNF-α production at the highest concentration used (2 mg/ml). No effect of the extract was observed in the absence of LPS.

As above values represent the means of ±s.d. of n=3. * denotes P<0.05 compared to LPS alone.

The Graph of FIG. 2 shows that TNF-α production is reduced in the order of 50%.

Further data have shown that preincubation for 24 hr with raw extract before the addition of LPS resulted in a more profound reduction (> than 85% at 10 μm) of TNF-α level. A comparison of preincubation times showed that an even more potent suppression occurred following 48 hr compared to 24 hr preincubation.

Therefore it may be concluded that raw cucumber (Cucumis sativus) extract is able to reduce the TNF-α production in human blood.

Moreover, the raw cucumber extract does not induce any adverse immune reaction. Therefore it may be administered as food supplement composition for a prolonged time without any side effect.

The prolonged use, i.e. for more than one week of a food supplement composition comprising raw cucumber extract could therefore reduce long-lasting inflammation states due to high TNF-α value in blood.

Moreover, the preincubation experiments shows that a food supplement composition comprising raw cucumber extract could be advantageously administered for a long time as food supplement to prevent the inflammation status due to an high TNF-α value in blood.

Unexpectedly such effect is much more evident with raw cucumber extract than with ido-BR1 and therefore it is assumed that the administration of raw cucumber extract is much more effective than the administration of the isolated ido-BR1.

### Example 2

Analysis of anti-inflammatory properties of Gherkin extract in porcine aortic endothelial cells during LPS treatment.

The Endothelium is the primary sensor of physical and chemical changes in the bloodstream.

Several studies have suggested a key role of the endothelium in the pathophysiology of severe sepsis; during this process, LPS (lipopolysaccharide), a component of the bacterial was able to cause structural and functional alterations of the endothelial phenotype hesitating in endothelium dysfunction.

Increasing evidence indicate the pig as an excellent model for studying human disease, particularly cardiovascular physiopathology.

An in vitro model of inflammatory response with a primary culture of porcine Aortic Endothelial Cells simulated with LPS was prepared. LPS induced alterations of the expression of several genes.

Hemeoxygenase (HO-1): anti-inflammatory molecule (increase at 7 h)

TNF-alpha: pro-inflammatory cytokine, it is a pleiotropic molecule that plays a central role in inflammation, apoptosis, and immune system development. (increase at 24 h)

VCAM-1 is an immunoglobulin-like adhesion molecule expressed on activated endothelial cells (increase at 1, 7, 24 h)

Zonulin is a protein that modulates the permeability of tight junctions between cells (decrease at 7 and 24 h) including endothelial cells.

### Material and Methods:

Cell type: pAECs (porcine Aortic Endothelial Cells)

Stimulus: LPS (10 μg/ml) for 1, 7, 24 h

Test compounds: Gherkin fruit extract (2, 200, 2000 μg/ml)

Read-out: Morphological alterations

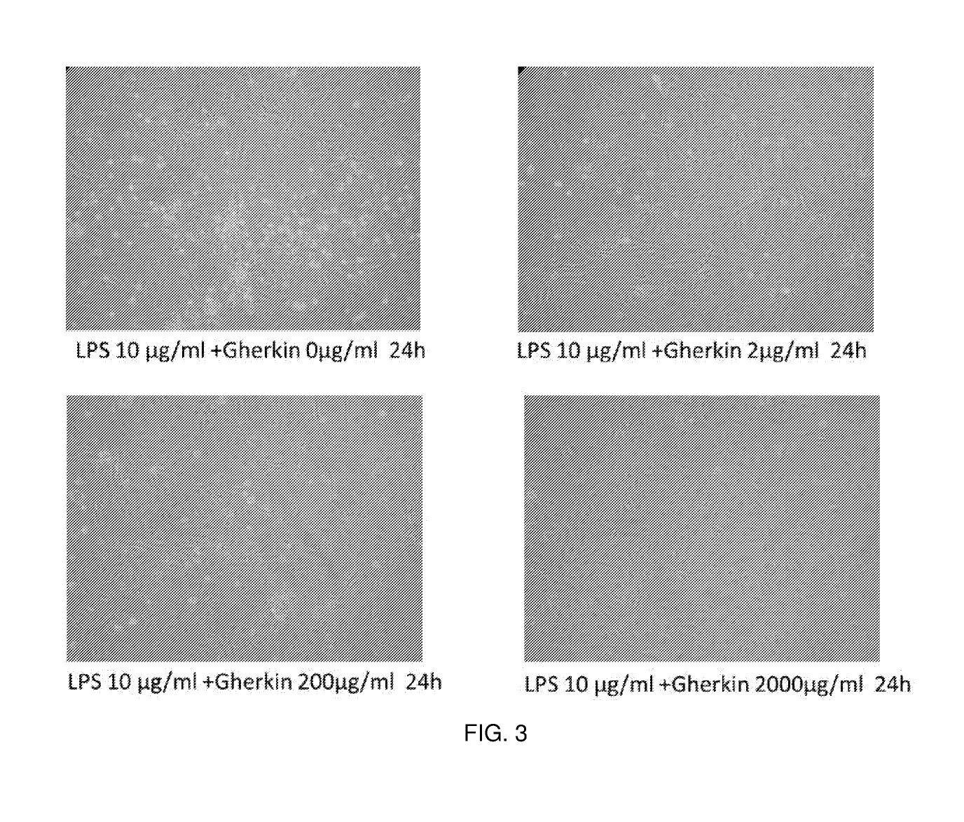

As can be seen in FIG. 3 LPS induced a spindle-shaped morphological change (stress fiber induction) and cell detachment Gherkin extract inhibited LPS-induced morphological changes and cell detachment in a dose dependent manner. The highest dose of gherkin alone exhibits signs of cytotoxicity.

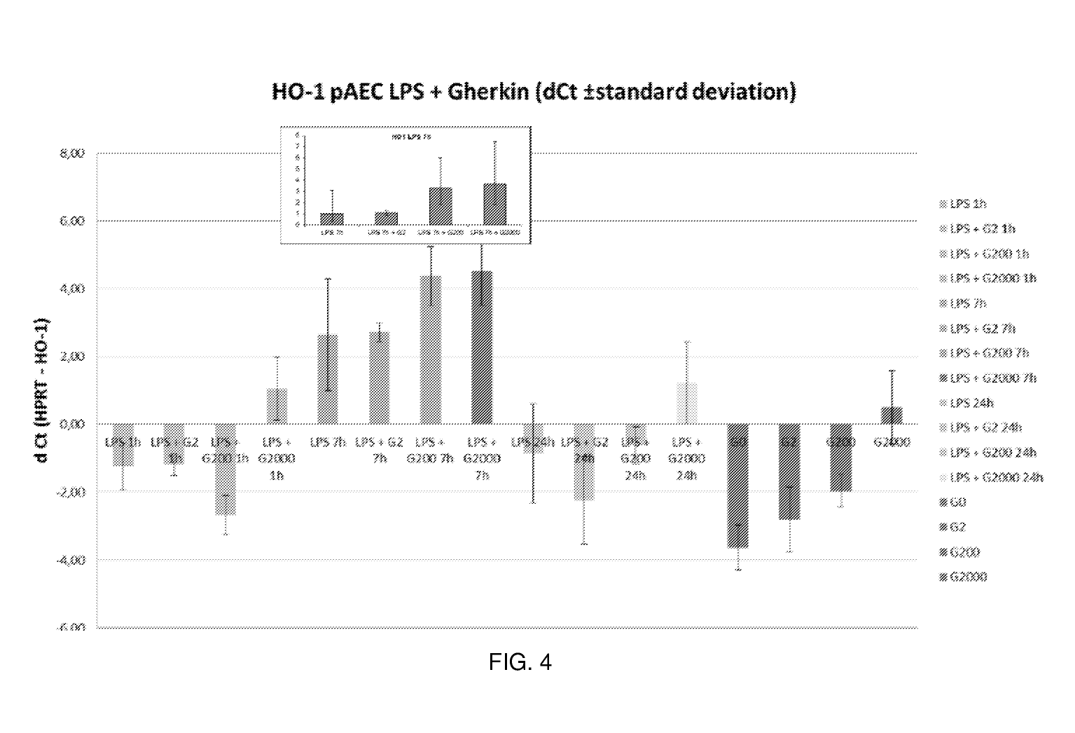

As can be seen in FIG. 4 gherkin extract induced HO-1 expression Gherkin treated cells showed higher level of protective HO-1 during LPS treatment than cells treated with only LPS.

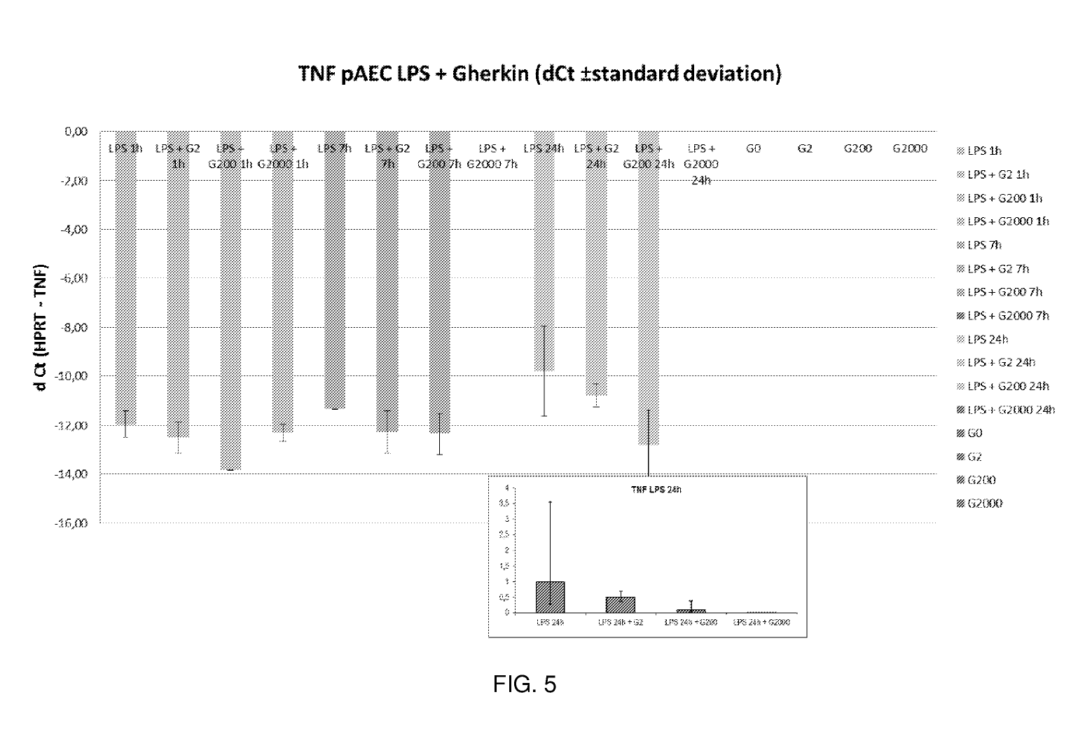

As can be seen in FIG. 5 gherkin extract inhibited LPS-induced TNF-alpha expression mainly at 24 h.

In conclusion Gherkin extract showed anti-inflammatory properties through attenuation of LPS-induce gene expression.

### Example 3

Field trials have been conducted to evaluate the effect and response of prolonged administration of gherkin extract.

The Capsule composition is the following:

a nucleus of 500 mg weight comprising 150 mg of dried raw extract of Cucumis sativus and silicon dioxide, microcrystalline cellulose, calcium phosphate, magnesium stearate
an external gastroresistant layer comprising Shellac (E904), polyvinylpirrolidone, ammonium bicarbonate, Carboxymethyl cellulose sodium, medium-chain triglyceride, silicon bioxide, titanium bioxide (E171).

In a first trail 20 athletes have been treated orally with 1 capsule 2 times a day at breakfast and lunch for 4 weeks.

Each athlete completed and signed a personal record with score and personal observation.

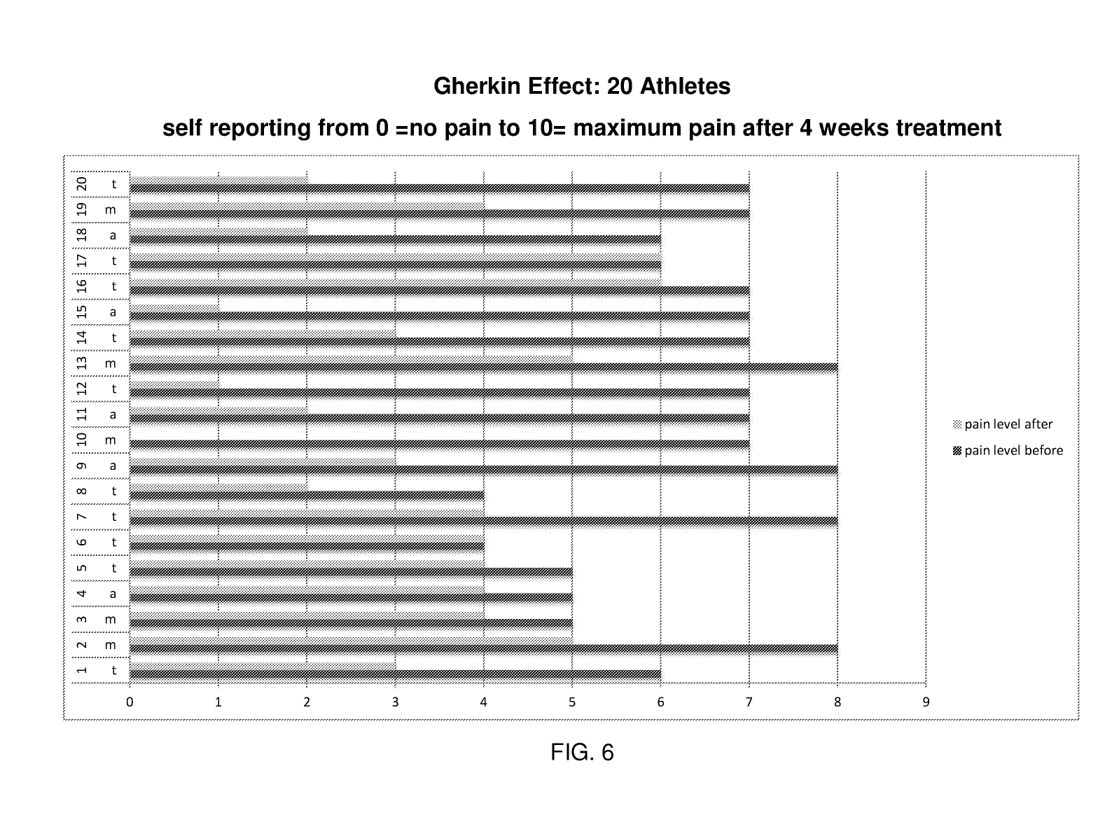

FIG. 6 shows a graph reporting the score given by the 20 athletes after a 4 week treatment.

Three type of affliction were studied: muscular inflammation (abbreviated in m in the graphs), tendons inflammations (abbreviated in t in the graphs) and articular inflammation (abbreviated in a in the graphs).

90% of the treated athletes reported positive effects with pain reduction, no need to use other anti-inflammatory and better performance. No negative or side effect were reported.

In a second trail 50 adult patients were treated with 1 capsule 2 times a day (breakfast lunch). The treatment period was 30 days.

A Symptomatic score was recorded: from 5 showing no effect to 10 showing complete symptoms remission.

3 Type of affliction were studied: general inflammation pain (abbreviated with g), muscular inflammation (abbreviated with (m)) and arthritis inflammation.

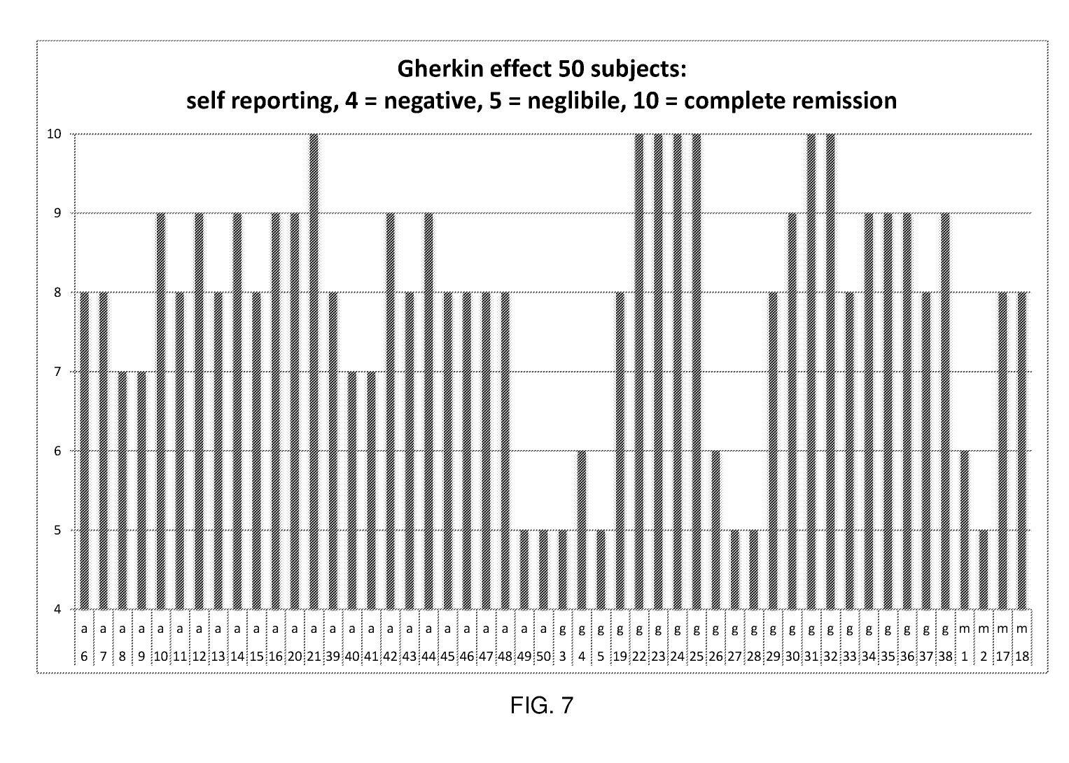

In FIG. 7 the results are reported.

Only 20% of the subjects found a negligible or minor effect, more than 80% of patients found a positive or strongly positive effect. A apsecial, strng positevly correlated effect was reported on articular arthritis pain and muscular tensive pain headache.

### Example 4

An analysis of anti-inflammatory properties of Gherkin fruit Extract in comparison with idoBR1 compound was made with procedures as those of Example 1.

Mouse Cells were tested. Cell type was dendritic cells D1 (mouse DC cell line), Stimulus was LPS (100 ng/ml).

Test compounds were the following:
Gherkin extract (1, 0.3, 0.1, 0.03 mg/ml)
ido BR1 (10, 3, 1, 0.3, 0.1 uM)

Read out: Cytokine secretion (IL12p40, TNF-alpha, IL-6)

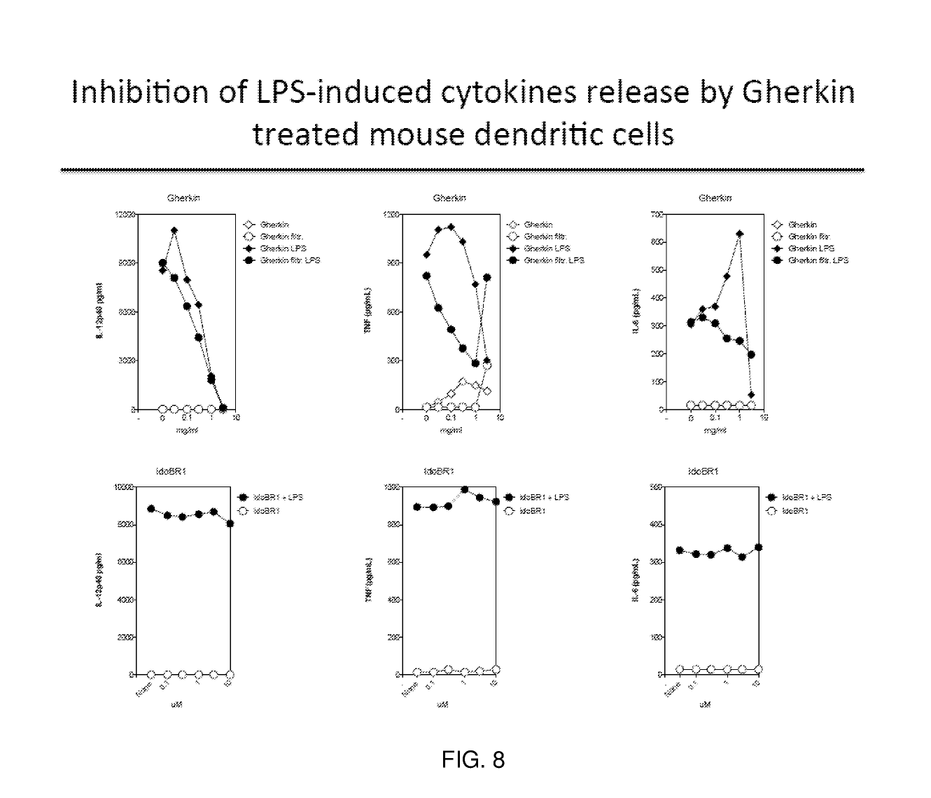

FIG. 8 shows the results when using the different quantities of gherkin fruit extract and idoBR1.

Similar tests were conducted on human moDC moDC (monocyte derived dendritic cells with a stimulus of LPS 100 ng/ml.

The test compounds were:

Gherkin extract (1, 0.3, 0.1, 0.03 mg/ml)

idoBR1 (1000, 300, 100, 30, 10 uM)

Read-out:

Cytokin secretion (IL12p40, TNF-alpha, IL-10

Allogenic naive T cell activation (IFN-g)

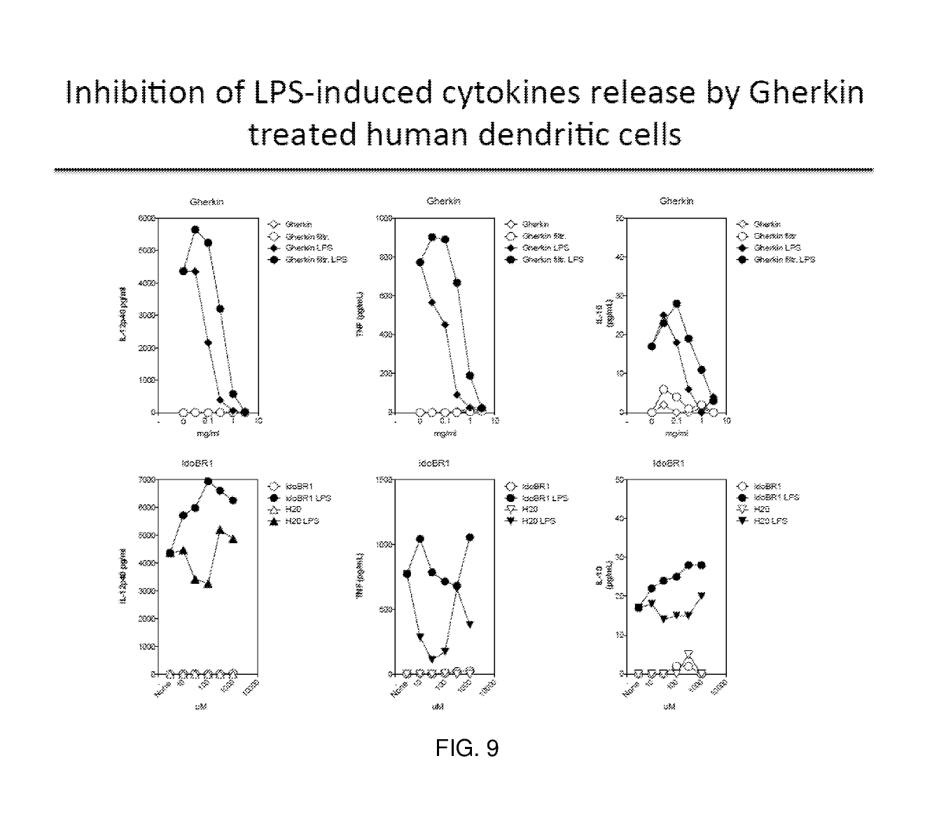

The results are shown in FIG. 9.

All the graphs show an improved efficacy of the raw extract from the fruit of cucumis sativus in comparison to an isolated ido_BR1.

1. Food supplement comprising an orally administrable dose form comprising a raw cucumber (Cucumis sativus) extract having an amount capable of reducing the TNF-alpha level in a subject.

2. (canceled)

3. (canceled)

4. Food supplement according to claim 1, comprising between 5 to 200 mg of the raw cucumber (Cucumis sativus) extract.

5. Food supplement according to claim 1, comprising between 10 to 150 mg of the raw cucumber (Cucumis sativus) extract.

6. Food supplement according to claim 1, wherein the dose form further comprises a substance chosen from the group consisting of silicon dioxide, cellulose, calcium phosphate, magnesium stearate.

7. Food supplement according to claim 1, characterized in that it comprises a nucleus comprising the raw extract and an external layer.

8. Food supplement according to claim 7, wherein the external layer is a gastro-resistant layer.

9. Food supplement according to claim 8, wherein the gastro-resistant layer comprise a substance chosen from the group consisting of microcrystalline cellulose, saccharose, corn starch, lactose and a mixture thereof.

10. Food supplement according to claim 7, wherein the nucleus and/or the external layer comprises a physiologically acceptable excipient selected from the group consisting of suspending agents, glidants, filming agents, plasticizing agents and mixture thereof.

11. Food supplement according to claim 9, wherein the filming agents are selected from alkyl cellulose hydroxyalkyl cellulose, polyvinylpyrrolidone (PVP), polyvinyl alcohol (PVA) and a mixture thereof.

12. Food supplement according to any of claim 9, wherein the external gastro-resistant layer comprises polymers and/or copolymers of acrylic acid and/or of methacrylic acids or salts thereof.

13. Food supplement according to claim any of claim 10, wherein the external gastro-resistant layer comprises sodium carboxymethylcellulose.

14. Food supplement according to claim 1, wherein it is an extract from cucumis sativus fruit.

15. A method of reducing TNF-alpha level in a subject, the method comprising:
orally administering to a subject a food supplement comprising a raw cucumber (Cucumis sativus) extract in an amount sufficient to reduce the TNF-alpha level in the subject; and
reducing the TNF-alpha level in the subject;
wherein oral administration is for more than one week.

16. The method of claim 15, further comprising treating a TNF mediated inflammatory disease.

17. The method of claim 16, wherein the TNF mediated inflammatory disease is one of adult respiratory distress syndrome, asthma, autoimmune disease, cachexia, a chronic pulmonary inflammatory disease, congestive heart failure, Crohn's disease, endotoxic shock, ENL in leprosy, fibrotic disease, graft rejection, hemodynamic shock, hyperoxic alveolar injury, inflammation, muscular inflammation, tendon inflammation, articular inflammation, malaria, meningitis, multiple sclerosis, mycobacterial infection, an oncogenic or cancerous condition, opportunistic infection in AIDS, osteoarthritis, post ischemic reperfusion injury, psoriasis, radiation damage, rheumatoid arthritis, rheumatoid spondylitis, septic shock, sepsis, sepsis syndrome, systemic lupus erythrematosis or ulcerative colitis.

18. The method of claim 15, wherein the effective amount is in the range of 5 to 500 mg of the raw cucumber extract per kilogram body weight.

19. The new method of claim 15, wherein the effective amount is in the range of 10 to 150 mg of the raw cucumber extract per kilogram body weight.

20. The method of claim 15, wherein the TNF-alpha is reduced by about 50%.
